(19) United States
(12) Patent Application Publication
(10) Pub. No.: US 2014/0037742 A1
(43) Pub. Date: Feb. 6, 2014

(54) ALGINATE MICROPARTICLES AND METHODS OF USING THE SAME

(71) Applicant: Melissa Fagan, San Diego, CA (US)

(72) Inventor: Melissa Fagan, San Diego, CA (US)

(21) Appl. No.: 13/954,784

(22) Filed: Jul. 30, 2013

Related U.S. Application Data
(60) Provisional application No. 61/678,046, filed on Jul. 31, 2012.

(51) Int. Cl. A61K 9/50; A61K 38/51; A61K 45/06; A61K 33/38

(52) U.S. Cl. CPC A61K 9/5036; A61K 33/38; A61K 38/51; A61K 45/06
USPC 424/493; 424/618; 424/94.5

(57) ABSTRACT

By combining the anti-bacterial effects of silver sulfadiazine and silver nanoparticles with the absorption and slow-release capabilities of alginate, a product was created that can be used to heal and protect deep wounds. These microparticles can have a greater surface area to volume ratio. This, in turn, can permit the particles to have a larger area of exposure to bacterial colonies, thereby increasing antimicrobial activity. Additionally, engineered microparticles also may benefit from the ability to conform to the shape of the wound.

### RELATED APPLICATIONS

The present application claims priority to U.S. Provisional Application Ser. No. 61/678,046, filed Jul. 31, 2012, which is hereby incorporated by reference in its entirety.

### FIELD

The present application generally relates to alginate microparticles comprising one or more substances such as antiseptics, antibiotics or bacteriostatics, and to methods of using the same.

### BACKGROUND

Alginates are polysaccharides isolated from brown algae and are the primary components of the cell wall and the extracellular matrix. Alginic acid and its salts, alginates, are widely used in numerous aspects of human life due to their ability to emulsify, jellify, thicken and stabilize industrial products and food. Today, alginic acid and alginates are also widely used in the medical world to deliver reagents and absorb bodily fluids.

Antibiotics are substances or medications that inhibit the growth of or destroy microorganisms. Bacteriostatics inhibit the growth of bacteria without necessarily destroying the organism. Resistance of microorganisms, particularly bacteria, is a serious concern due to the growing number of antibiotics to which bacteria are resistant.

Described herein are novel compositions and methods related to the use of alginate microparticles for the delivery of substances such as antiseptics, antibiotics, bacteriostatics and other antimicrobial substances.

### SUMMARY OF THE INVENTION

Generally described herein are compositions and methods related to the use of alginate microparticles comprising antiseptics, such as antibiotics, bacteriostatics or other antimicrobial substances. The compositions and methods can be used in some embodiments to deliver such substances while avoiding or minimizing potential toxicity issues and/or while avoiding or reducing the need to use antibiotics that are subject to or potentially susceptible to resistance.

Disclosed herein are compositions of matter comprising an alginate microparticle and silver. In some embodiments the alginate microparticle has a diameter of less than 10 μm. In some aspects the microparticle population has a mean diameter less than 10 μm. In some aspects the microparticle population has a mean diameter of about 7 μm. As used herein, the phrase ‘about’ a given values is defined as said value plus and minus 10% of said value. In some aspects the alginate particle comprises and alginate-degrading chemical, such as an alginate degrading enzyme, for example an alginate lyase.

Disclosed herein are methods of delivery of an antiseptic, antibiotic or bacteriostatic. In some aspects the methods comprise providing a composition comprising a microparticle population. In some aspects the microparticle population comprises microparticles comprising an antiseptic, such as an antibiotic or bacteriostatic. In some embodiments the microparticle comprises alginate and silver. In some aspects a microparticle population has a mean diameter of less than 10 μm. In some aspects the antiseptic comprises silver sulfadiazine, silver nano particles and/or colloidal silver. In some aspects the delivery comprises sustained release of said antiseptic, such as an antibiotic or bacteriostatic from said alginate microparticle. In some aspects the sustained release continues for not less than 24 hours. In some aspects the sustained release continues for not less than 48 hours. In some aspects the sustained release continues for not less than 72 hours. In some aspects the sustained release continues for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 hours.

Some aspects of the disclosed methods comprise contacting said composition to a wound or infection site of a subject needing antibacterial or bacteriostatic treatment at a wound site.

In some aspects an alginate lyase is provided. In some aspects an alginate lyase is contacted to a wound site. In some aspects the lyase releases substantially all of said antiseptic, antibiotic or bacteriostatic from said microparticle population.

Disclosed herein are methods of controlling a duration of exposure to an antiseptic, antibiotic or bacteriostatic in a wound site of a subject. In some aspects these methods comprise providing a composition comprising a microparticle population; wherein said microparticle population comprises microparticles comprising an antiseptic, such as an antibiotic or bacteriostatic, and an alginate microparticle encompassing said antiseptic, antibiotic or bacteriostatic, and wherein said microparticle population has a mean diameter of less than 10 μm; measuring an amount of time during which a subject is to be exposed to said antiseptic, antibiotic or bacteriostatic, and providing at the passage of said duration of time an alginate lyase to said wound site of said subject.

In some aspects the antiseptic, antibiotic or bacteriostatic comprises silver. In some aspects the antiseptic, antibiotic or bacteriostatic comprises silver sulfadiazine, colloidal silver and/or silver nano particle. In some aspects concentration of said antiseptic, antibiotic or bacteriostatic is between 20 μg/ml and 60 μg/ml.

### BRIEF DESCRIPTION OF THE DRAWINGS

FIG. 1 illustrates strands of a 2% (wt/vol) solution of sodium alginate stained with Safranin O prior to emulsification and internal gelation. Photo was taken with a photomicroscope under a 10× zoom feature.

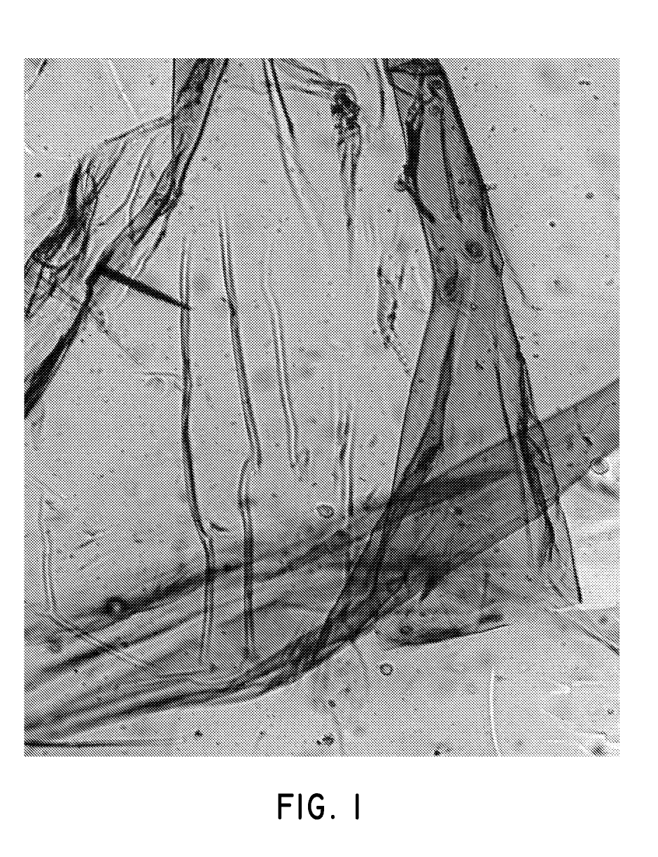

FIG. 2 illustrates the separation of alginate microparticles containing SSD from the oil phase during emulsification and internal gelation. The aggregated microparticles can be seen as a gray layer suspended in a 50 mM calcium chloride solution.

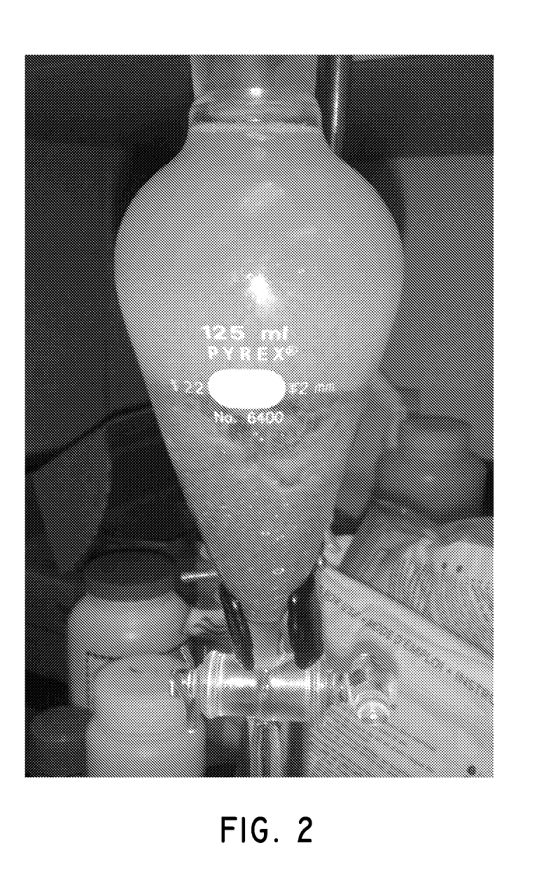

FIG. 3 illustrates a sample setup of antimicrobial testing on Staphylococcus epidermidis. Each disc was saturated with different test materials and zones of inhibition were measured after plates were incubated for 24 and 48 hours.

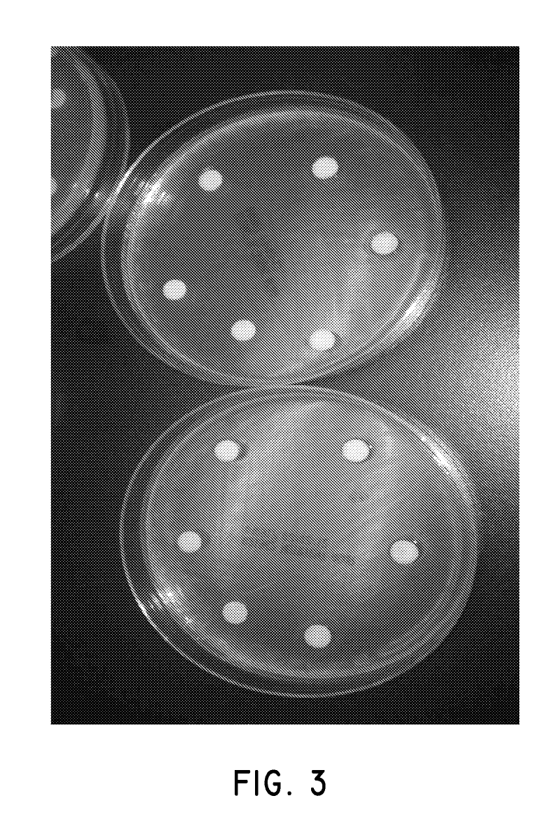

FIG. 4 illustrates strands of a 2% solution of sodium alginate mixed with silver sulfadiazine and stained with Safranin O prior to emulsification and internal gelation. Photo was taken with a photomicroscope under a 10× zoom feature.

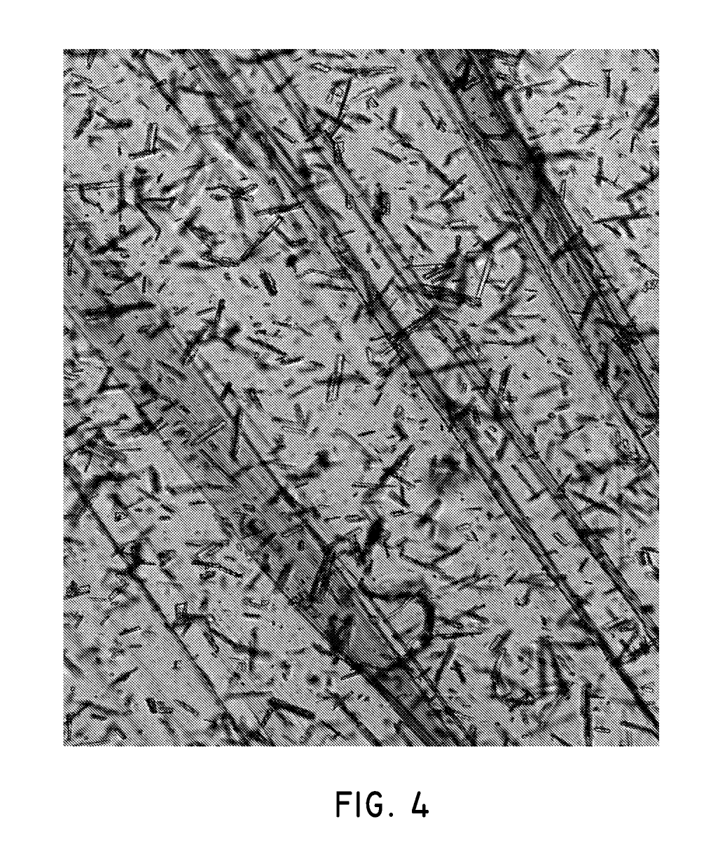

FIG. 5 illustrates engineered alginate microparticles containing SSD stained with Safranin O. Visible amounts of SSD can be seen encapsulated within the microparticles. Photo was taken with a photomicroscope under a 4× zoom feature.

FIG. 6 illustrates the synthesized alginate microparticles stained with Safranin O after two weeks in 4° C. storage. Photo was taken with a photomicroscope under a 10× zoom feature.

FIG. 7 illustrates engineered alginate microparticles containing SSD stained with Safranin O. As seen in FIG. 5, visible amounts of SSD can be seen encapsulated within the microparticles. Photo was taken with a photomicroscope under a 20× zoom feature.

FIG. 8 illustrates synthesized alginate microparticles stained with Safranin O after two weeks in 4° C. storage. Photo was taken with a photomicroscope under a 20× zoom feature.

FIG. 9 illustrates the mean diameter of alginate microparticles alone and alginate microparticles containing SSD. Error bars indicate Standard Error. Alginate microparticles containing SSD were shown to be significantly larger when compared to alginate particles alone (p<0.05), determined significant using a two-tailed unpaired t-test.

FIG. 10 illustrates the mean density of alginate microparticles alone and alginate microparticles containing SSD. Error bars indicate Standard Error. Alginate microparticles containing SSD were shown to be significantly more dense when compared to alginate particles alone (p<0.1), determined significant using a two-tailed unpaired t-test.

FIG. 11 illustrates the mean zones of inhibition of Staphylococcus epidermidis when incubated with various test materials at 24 hours, data shown +/−standard error bars. Alginate microparticles containing silver sulfadiazine (SSD) were shown to have significantly larger zones of inhibition when compared to alginate microparticles alone (p<0.001) determined using a one-tailed unpaired t-test. Alginate microparticles containing SSD had no significant difference when compared to SSD (p<0.05), indicated with a dot.

FIG. 12 illustrates the mean zones of inhibition of S. epidermidis when incubated with various test materials at 48 hours, data shown +/−standard error. Alginate microparticles containing SSD were shown to have significantly larger zones of inhibition when compared to alginate microparticles alone (p<0.001) determined using a one-tailed unpaired t-test. Alginate microparticles containing SSD had no significant difference when compared to SSD (p<0.05), indicated with a dot.

FIG. 13 illustrates a comparison of shows a comparison of mean zones of inhibition of S. epidermidis when incubated with various test materials at 24 and 48 hours. The results are shown with the difference in the baseline negative control (alginate microparticles) from 24 to 48 hours removed, results shown ±standard error bars. Alginate microparticles containing silver sulfadiazine (SSD) were shown to have a significant increase in the zone of inhibition from 24 to 48 hours (p<0.05*). Alginate microparticles containing SSD also showed a significantly larger zone of inhibition when compared to SSD (p<0.05) at 48 hours. In all cases, * and • represent significance using a one tailed unpaired t-test.

FIG. 14 illustrates the mean zones of inhibition of S. epidermidis when incubated with alginate microparticles containing silver sulfadiazine (SSD) and SSD alone at 24-96 hours, data shown ±standard error bars. Alginate microparticles containing silver sulfadiazine (SSD) were shown to have significantly larger zones of inhibition when compared to SSD alone at 48, 72, and 96 hours (p<0.05*). * indicates significance using a two-tailed unpaired t-test.

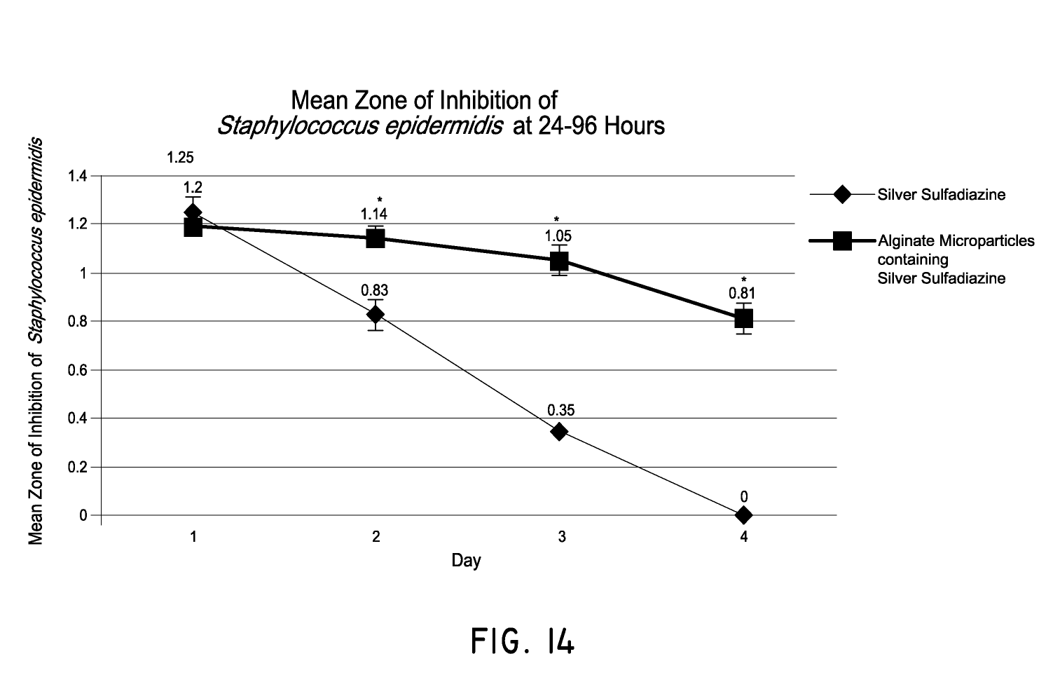

FIG. 15 illustrates shows the mean zone of inhibition of Staphylococcus epidermidis when incubated with alginate microparticles containing silver sulfadiazine (SSD) with and without alginate lyase. Alginate microparticles containing SSD with alginate lyase produced a significantly larger zone of inhibition at 24 hours (p<0.001*) in comparison to alginate microparticles containing SSD without lyase. * Indicates the significance using a one-tailed unpaired t-test.

FIG. 16 illustrates the mean zone of inhibition of S. epidermidis when incubated with alginate microparticles containing silver sulfadiazine (SSD) with and without alginate lyase. Alginate microparticles containing SSD with alginate lyase produced a significantly larger zone of inhibition at 48 hours (p<0.001*) in comparison to alginate microparticles containing SSD without lyase. * indicates the significance using a one-tailed unpaired t-test.

### DETAILED DESCRIPTION OF THE PREFERRED EMBODIMENT

Bacterial resistance is a growing issue in the modern world, causing the medical community to search for alternative methods to fight infection in chronic wounds. Embodiments herein generally relate to microparticles, preferably microparticles at least comprising alginate, comprising substances that can be used to for the treatment of wounds and/or the treatment or prevention of infections.

Any suitable antimicrobial substance or medicament for wounds can be used with the alginate microparticles. The particles can have a diameter of about 500 nanometers to about 10 micrometers, such as 500 nm, 600 nm, 700 nm, 800 nm, 900 nm, 1 μm, 1.1 μm, 1.2 μm, 1.3 μm, 1.4 μm, 1.5 μm, 1.6 μm, 1.7 μm, 1.8 μm, 1.9 μm, 2.0 μm, 2.1 μm, 2.2 μm, 2.3 μm, 2.4 μm, 2.5 μm, 2.6 μm, 2.7 μm, 2.8 μm, 2.9 μm, 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, 5.0 μm, 5.1 μm, 5.2 μm, 5.3 μm, 5.4 μm, 5.5 μm, 5.6 μm, 5.7 μm, 5.8 μm, 5.9 μm, 6.0 μm, 6.1 μm, 6.2 μm, 6.3 μm, 6.4 μm, 6.5 μm, 6.6 μm, 6.7 μm, 6.8 μm, 6.9 μm, 7.0 μm, 7.1 μm, 7.2 μm, 7.3 μm, 7.4 μm, 7.5 μm, 7.6 μm, 7.7 μm, 7.8 μm, 7.9 μm, 8.0 μm, 8.1 μm, 8.2 μm, 8.3 μm, 8.4 μm, 8.5 μm, 8.6 μm, 8.7 μm, 8.8 μm, 8.9 μm, 9.0 μm, 9.1 μm, 9.2 μm, 9.3 μm, 9.4 μm, 9.5 μm, 9.6 μm, 9.7 μm, 9.8 μm, 9.9 μm, or 10 μm.

A population of particles may have an average diameter ranging from any subrange or specific value there between, such as a range of the smaller of 500 nm, 600 nm, 700 nm, 800 nm, 900 nm, 1 μm, 1.1 μm, 1.2 μm, 1.3 μm, 1.4 μm, 1.5 μm, 1.6 μm, 1.7 μm, 1.8 μm, 1.9 μm, 2.0 μm, 2.1 μm, 2.2 μm, 2.3 μm, 2.4 μm, 2.5 μm, 2.6 μm, 2.7 μm, 2.8 μm, 2.9 μm, 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, 5.0 μm, 5.1 μm, 5.2 μm, 5.3 μm, 5.4 μm, 5.5 μm, 5.6 μm, 5.7 μm, 5.8 μm, 5.9 μm, 6.0 μm, 6.1 μm, 6.2 μm, 6.3 μm, 6.4 μm, 6.5 μm, 6.6 μm, 6.7 μm, 6.8 μm, 6.9 μm, 7.0 μm, 7.1 μm, 7.2 μm, 7.3 μm, 7.4 μm, 7.5 μm, 7.6 μm, 7.7 μm, 7.8 μm, 7.9 μm, 8.0 μm, 8.1 μm, 8.2 μm, 8.3 μm, 8.4 μm, 8.5 μm, 8.6 μm, 8.7 μm, 8.8 μm, 8.9 μm, 9.0 μm, 9.1 μm, 9.2 μm, 9.3 μm, 9.4 μm, 9.5 μm, 9.6 μm, 9.7 μm, 9.8 μm, or 9.9 μm, to a second larger value such as 600 nm, 700 nm, 800 nm, 900 nm, 1 μm, 1.1 μm, 1.2 μm, 1.3 μm, 1.4 μm, 1.5 μm, 1.6 μm, 1.7 μm, 1.8 μm, 1.9 μm, 2.0 μm, 2.1 μm, 2.2 μm, 2.3 μm, 2.4 μm, 2.5 μm, 2.6 μm, 2.7 μm, 2.8 μm, 2.9 μm, 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, 5.0 μm, 5.1 μm, 5.2 μm, 5.3 μm, 5.4 μm, 5.5 μm, 5.6 μm, 5.7 μm, 5.8 μm, 5.9 μm, 6.0 μm, 6.1 μm, 6.2 μm, 6.3 μm, 6.4 μm, 6.5 μm, 6.6 μm, 6.7 μm, 6.8 μm, 6.9 μm, 7.0 μm, 7.1 μm, 7.2 μm, 7.3 μm, 7.4 μm, 7.5 μm, 7.6 μm, 7.7 μm, 7.8 μm, 7.9 μm, 8.0 μm, 8.1 μm, 8.2 μm, 8.3 μm, 8.4 μm, 8.5 μm, 8.6 μm, 8.7 μm, 8.8 μm, 8.9 μm, 9.0 μm, 9.1 μm, 9.2 μm, 9.3 μm, 9.4 μm, 9.5 μm, 9.6 μm, 9.7 μm, 9.8 μm, 9.9 μm, or 10 μm. In some instances the particles can have diameters ranging from about 3 micrometers to about 6 micrometers, for example. In some instances, the particles can be larger than 10 micrometers, for example, when using non-silver substances.

Alginate microparticles can be obtained through a variety of methods, one of the most prominent being emulsification and internal gelation. In some embodiments of the instant technology the particles created can have a diameter in nanometer to micrometer size. For example, in some embodiments the particles can be between about 500 nanometers and 10 μm, or any value or subrange between. In some particular aspects the instant technology relates to alginate particles having a diameter of 10 μm or less.

Non-limiting examples of substances that can be delivered using the alginate particles described herein include antiseptics, antibiotics and bacteriostatics. As an example heavy metals such as silver (nanoparticulate silver, silver sulfadiazine, colloidal silver, etc.), honey (e.g., Manuka honey), and other antimicrobial substances may be delivered in some embodiments.

In some embodiments, the compositions disclosed herein are delivered to a subject in need thereof, e.g., a subject having a wound. As used herein, the term “subject” includes warm-blooded animals, preferably mammals, including humans. In a preferred embodiment, the subject is a primate. In an even more preferred embodiment, the subject is a human.

Antiseptics are substances that prevent the growth of disease-causing organisms. Antiseptics work to prevent and treat conditions such as serious wound infections by preventing the reproduction of bacteria. A partial list of exemplary antiseptics includes “surgical alcohols” such as ethanol (60-90%), 1-propanol (60-70%) and 2-propanol/isopropanol (70-80%) or mixtures of these alcohols; iodine (tincture of iodine), cationic surfactants (benzalkonium chloride 0.05-0.5%, chlorhexidine 0.2-4.0% or octenidine dihydrochloride 0.1-2.0%); Quaternary ammonium compounds such as benzalkonium chloride (BAC), cetyl trimethylammonium bromide (CTMB), cetylpyridinium chloride (Cetrim, CPC) and benzethonium chloride (BZT), and related disinfectants chlorhexidine and octenidine; boric acid; ‘Brilliant Green’ triarylmethane dye; Chlorhexidine Gluconate alone or in lower concentrations in combination with other compounds, such as alcohols; hydrogen peroxide; iodine; iodine antiseptics containing povidone-iodine (an iodophor, complex of povidone, a water-soluble polymer, with triiodide anions I3−, containing about 10% of active iodine); Manuka Honey; Octenidine dihydrochloride and bis-(dihydropyridinyl)-decane derivative, 2-phenoxyethanol; Phenol (carbolic acid) compounds Sodium chloride, Sodium hypochlorite, and Calcium hypochlorite. Other examples are contemplated.

A subset of antiseptics includes antibiotics. Antibiotics often show microbicidal activity. Exemplary antibiotics useful in the embodiments disclosed herein include, but are not limited to, Aminoglycosides, Amikacin, Gentamicin, Kanamycin, Neomycin, Netilmicin, Tobramycin, Paromomycin, Spectinomycin, Ansamycins, Geldanamycin, Herbimycin, Rifaximin, streptomycin, Carbacephem, Loracarbef, Carbapenems, Ertapenem, Doripenem, Imipenem/Cilastatin, Meropenem, Cephalosporins, Cefadroxil, Cefazolin, Cefalotin or Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, Ceftobiprole, Glycopeptides, Teicoplanin, Vancomycin, Telavancin, Lincosamides, Clindamycin, Lincomycin, Lipopeptide, Daptomycin, Macrolides, Azithromycin, Clarithromycin, Dirithromycin, Erythromycin, Roxithromycin, Troleandomycin, Telithromycin, Spiramycin, Monobactams, Aztreonam, Nitrofurans, Furazolidone, Nitrofurantoin, Oxazolidonones, Linezolid, Posizolid, Radezolid, Torezolid, Penicillins, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, Amoxicillin/clavulanate, Ampicillin/sulbactam, Piperacillin/tazobactam, Ticarcillin/clavulanate, Polypeptides, Bacitracin, Colistin, Polymyxin B, Quinolones, Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin, Temafloxacin, Sulfonamides, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole) (TMP-SMX), Sulfonamidochrysoidine (archaic), Tetracyclines, Demeclocycline, Doxycycline, Minocycline, Oxytetracycline, Tetracycline, Clofazimine, Dapsone, Capreomycin, Cycloserine, Ethambutol, Ethionamide, Isoniazid, Pyrazinamide, Rifampicin/Rifampin, Rifabutin, Rifapentine, Streptomycin, Others, Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole, or Trimethoprim.

Antiseptics may also include bacteriostatics. Bacteriostatics block bacterial or microbial growth. Exemplary bacteriostatics useful in the embodiments disclosed herein include, but are not limited to, tetracyclines such as Tetracycline, Chlortetracycline, Oxytetracycline, Demeclocycline, Doxycycline, Lymecycline, Meclocycline, Methacycline, Minocycline, Rolitetracycline, and Tigecycline; Sulfamethoxazole, Sulfisomidine (also known as sulfaisodimidine), Sulfadiazine, Sulfacetamide, Sulfadoxine, Dichlorphenamide (DCP), trimethoprim, chloramphenicol, lincomycin, clindamycin, Azithromycin, Clarithromycin, Dirithromycin, Erythromycin, Roxithromycin, Telithromycin, Carbomycin A, Josamycin, Kitasamycin, Midecamycin/midecamycin acetate, Oleandomycin, Solithromycin, Spiramycin, Troleandomycin, Tylosin/tylocine, Telithromycin, Cethromycin, Solithromycin, Spiramycin, Ansamycin, Oleandomycin, Carbomycin, and Tylosin.

Antibiotics, antiseptics or bacteriostatic as contemplated herein may be used at a broad range of concentrations, such as a concentration of as low as or lower than 0.5 μg/ml, to 0.6 μg/ml, 0.7 μg/ml, 0.8 μg/ml, 0.9 μg/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 21 μg/ml, 22 μg/ml, 23 μg/ml, 24 μg/ml, 25 μg/ml, 26 μg/ml, 27 μg/ml, 28 μg/ml, 29 μg/ml, 30 μg/ml, 31 μg/ml, 32 μg/ml, 33 μg/ml, 34 μg/ml, 35 μg/ml, 36 μg/ml, 37 μg/ml, 38 μg/ml, 39 μg/ml, 40 μg/ml, 41 μg/ml, 42 μg/ml, 43 μg/ml, 44 μg/ml, 45 μg/ml, 46 μg/ml, 47 μg/ml, 48 μg/ml, 49 μg/ml, 50 μg/ml, 51 μg/ml, 52 μg/ml, 53 μg/ml, 54 μg/ml, 55 μg/ml, 56 μg/ml, 57 μg/ml, 58 μg/ml, 59 μg/ml, 60 μg/ml, 61 μg/ml, 62 μg/ml, 63 μg/ml, 64 μg/ml, 65 μg/ml, 66 μg/ml, 67 μg/ml, 68 μg/ml, 69 μg/ml, 70 μg/ml, 71 μg/ml, 72 μg/ml, 73 μg/ml, 74 μg/ml, 75 μg/ml, 76 μg/ml, 77 μg/ml, 78 μg/ml, 79 μg/ml, 80 μg/ml, 81 μg/ml, 82 μg/ml, 83 μg/ml, 84 μg/ml, 85 μg/ml, 86 μg/ml, 87 μg/ml, 88 μg/ml, 89 μg/ml, 90 μg/ml, 91 μg/ml, 92 μg/ml, 93 μg/ml, 94 μg/ml, 95 μg/ml, 96 μg/ml, 97 μg/ml, 98 μg/ml, 99 μg/ml, 100 μg/ml, 101 μg/ml, 102 μg/ml, 103 μg/ml, 104 μg/ml, 105 μg/ml, 106 μg/ml, 107 μg/ml, 108 μg/ml, 109 μg/ml, 110 μg/ml, 111 μg/ml, 112 μg/ml, 113 μg/ml, 114 μg/ml, 115 μg/ml, 116 μg/ml, 117 μg/ml, 118 μg/ml, 119 μg/ml, 120 μg/ml, 121 μg/ml, 122 μg/ml, 123 μg/ml, 124 μg/ml, 125 μg/ml, 126 μg/ml, 127 μg/ml, 128 μg/ml, 129 μg/ml, 130 μg/ml, 131 μg/ml, 132 μg/ml, 133 μg/ml, 134 μg/ml, 135 μg/ml, 136 μg/ml, 137 μg/ml, 138 μg/ml, 139 μg/ml, 140 μg/ml, 141 μg/ml, 142 μg/ml, 143 μg/ml, 144 μg/ml, 145 μg/ml, 146 μg/ml, 147 μg/ml, 148 μg/ml, 149 μg/ml, 150 μg/ml, 151 μg/ml, 152 μg/ml, 153 μg/ml, 154 μg/ml, 155 μg/ml, 156 μg/ml, 157 μg/ml, 158 μg/ml, 159 μg/ml, 160 μg/ml, 161 μg/ml, 162 μg/ml, 163 μg/ml, 164 μg/ml, 165 μg/ml, 166 μg/ml, 167 μg/ml, 168 μg/ml, 169 μg/ml, 170 μg/ml, 171 μg/ml, 172 μg/ml, 173 μg/ml, 174 μg/ml, 175 μg/ml, 176 μg/ml, 177 μg/ml, 178 μg/ml, 179 μg/ml, 180 μg/ml, 181 μg/ml, 182 μg/ml, 183 μg/ml, 184 μg/ml, 185 μg/ml, 186 μg/ml, 187 μg/ml, 188 μg/ml, 189 μg/ml, 190 μg/ml, 191 μg/ml, 192 μg/ml, 193 μg/ml, 194 μg/ml, 195 μg/ml, 196 μg/ml, 197 μg/ml, 198 μg/ml, 199 μg/ml, or 200 μg/ml, or a concentration greater than 200 μg/ml.

Silver sulfadiazine and silver nano-crystalline structures belong to a class of antiseptic medicines. Recently, Silver sulfadiazine and silver nano-crystalline antiseptics have gained interest as a means of controlling infection due to a lack of resistance among bacteria. Delivery of silver is critical to its efficacy; however exposure to silver too rapidly comes at a high cost to the subject. Cell death and absorption of silver into the metabolism are common side effects of mass silver exposure. Thus, ensuring the balance between the antimicrobial properties of silver and safety of the subject is important to the success of the treatment.

Silver is an example of an antimicrobial that does not have the same issues with resistance associated with antibiotics. The success of silver depends upon a dose that maximizes antimicrobial activity but minimizes toxicity. Therefore alginate microparticles, encapsulating silver antiseptics, were engineered through emulsification and internal gelation with the objective of minimizing the downsides of rapid silver deactivation and toxicity. It should be noted that although various forms of silver are specifically discussed and detailed herein, other antimicrobial substances can be substituted for or used in place of silver. One such example of a particular antimicrobial substance is honey, for example, Manuka honey. One or more additional antiseptics, such as an antibiotic or bacteriostatic including but not limited to those disclosed herein, may be provided in addition to the silver antiseptic disclosed herein, either as a constituent of an alginate particle or provided accompanying an alginate particle.

In some embodiments, the microparticles disclosed herein provide a molar ratio of silver to alginate of 1:1, 1:2, 1:3, 1:4 1:5, 1:6, 1:7, 1.8, 1:9, 1:10, 1:11, 1:12, 1:13, 1:14, 1:15, 1:16, 1:20, 19:1, 18:1, 17:1, 16:1, 15:1, 15:1, 14:1, 13:1, 12:1, 11:1; 10:1, 9:1, 8:1, 7:1, 6:1, 5:1, 4:1, 3:1, 2:1, or any ratio in between.

The particles described herein can be used to treat or prevent conditions, for example, infections or wounds. As defined herein, a wound refers to an injured tissue, through trauma or infection, for example, which results in an opening in the skin or other surface of a person or mammal. The particles and compositions comprising the particles can be applied or administered to a subject in need of such treatment or prevention topically, for example. The particles can be administered topically as part of a liquid, a gel, a salve, emulsion, ointment, and the like. The particles can be included with a bandage, a patch, dressing, and the like. The particles can be aerosolized and administered in any suitable manner. For example, the aerosolized particles can be administered to an eye, mucosa, the skin or surface of a wound.

Microparticles were synthesized with and without the encapsulation of silver sulfadiazine (SSD) and tested for their ability to inhibit the growth of Staphylococcus epidermidis, a substitute for the pathogenic Staphylococcus aureus. Research was also conducted to determine stability, size and average density of the microparticles.

The manufactured microparticles were shown to be stable over time with regard to particle size, density and appearance. Concordant with previously mentioned research, the mean diameter of alginate microparticles alone and alginate microparticles containing SSD was 3.3±3.2 μm (standard deviation; “SD”) and 6.9±6.5 μm (SD), respectively. This number was shown to have little variability through batches. Similar consistent data was demonstrated for density, which had a mean of 6.7±3.3×10⁵ (SD) for alginate microparticles alone and 8.9±3.9×10⁵ (SD) for alginate microparticles containing SSD. Photographic analysis revealed that created particles were roughly symmetrical and that visible amounts of SSD could be seen encapsulated within the alginate microparticles. In some embodiments the microparticles can include a substance in addition to alginate. In some specific embodiments the microparticles can specifically exclude chitin as an additional material.

Results demonstrated that the alginate microparticles had an average size of between 1-10 μm and were stable over a period of two and three weeks with regard to density, particle size and appearance. Most of the microparticles were spherical in shape and had an average size of between 1-10 μm. Antimicrobial testing at 24 and 48 hours showed that alginate microparticles containing SSD produced significantly increased antimicrobial activity when compared to alginate microparticles alone (p<0.001). Results demonstrated a significant increase in the zone of inhibition of alginate microparticles containing SSD from 24 to 48 hours (p<0.05 vs. SSD alone). The release continued through 96 hours (p<0.001 vs. SSD alone), illustrating the ability of alginate to provide a continual release of encapsulated agents.

Further experiments were conducted to investigate release of SSD from alginate microparticles. Pre-treatment with alginate-lyase showed a decrease in anti-microbial activity from 24 to 48 hours, suggesting that alginate lyase breaks down the microparticles causing immediate release of SSD (p<0.05).

The synthesized microparticles were tested for their antimicrobial activity against Staphylococcus epidermidis, a substitute for the pathogenic Staphylococcus aureus. Results showed that alginate microparticles containing SSD produced significantly increased antimicrobial activity when compared to alginate microparticles alone, at both 24 and 48 hours. Alginate microparticles containing SSD (undiluted) had increased antimicrobial properties when compared to alginate microparticles containing SSD at dilution concentrations 1:10 and 1:100 at 24 and 48 hours. Therefore, alginate microparticles can be used to encapsulate and release SSD.

Antimicrobial testing at 24 hours showed no significant difference between the synthesized alginate microparticles containing SSD and SSD alone. At 48 hours, alginate microparticles containing SSD saw a significant increase in the zone of inhibition. SSD alone, however, saw a small reduction in antimicrobial activity at 48 hours. This may be explained by the ability of alginate microparticles to provide a continual release of encapsulated agents. This trend continued through 96 hours, reinforcing the ability of alginate to provide a continual release of encapsulated agents. Alginate particles protect encapsulated contents from premature degradation and offer a sustained release with time (Shanmugasundaram et al; 2008). This can be attributed to diffusion and enzymatic polymer matrix erosion. The sustained release protects the silver from being immediately converted to inactive silver compounds which occurs when silver is exposed to a wound environment. Results from antimicrobial analysis involving alginate lyase revealed that the engineered alginate microparticles containing SSD and lyase had a significantly increased zone of inhibition when compared to identical microparticles without lyase at both 24 and 48 hours. Antimicrobial activity of lyase infused alginate microparticles containing SSD did not increase from 24 to 48 hours. The enzyme (alginate lyase) breaks the carbon-oxygen bonds of the protective alginate layer, quickly releasing the silver into surrounding areas within the first 24 hours.

The synthesized microparticles were tested for their antimicrobial activity against Staphylococcus epidermidis, a substitute for the pathogenic Staphylococcus aureus. Results showed that alginate microparticles containing SSD produced significantly increased antimicrobial activity when compared to alginate microparticles alone, at both 24 and 48 hours. Furthermore, alginate microparticles (undiluted) had increased antimicrobial properties when compared to alginate microparticles containing SSD at dilution concentrations 1:10 and 1:100 at 24 and 48 hours. Both these findings reinforce the notion that alginate microspheres can be used to encapsulate and release SSD.

Moreover, results from antimicrobial analysis with alginate lyase, showed a significantly increased zone of inhibition of alginate microparticles containing SSD when compared to identical microparticles without the incorporation of alginate lyase at both 24 and 48 hours. Antimicrobial activity of alginate microparticles containing SSD and lyase did not increase from 24 to 48 hours. Once again, this can be explained by a sustained release theory. The enzyme—alginate lyase—breaks the carbon-oxygen bonds of the protective alginate layer, releasing the silver into surrounding areas within the first 24 hours and as such no further increase in the zone of inhibition occurred between 24 and 48 hours, whereas for microparticles that were untreated with the enzyme there was a more gradual release of SSD, increasing the zone of inhibition further between 24 and 48 hours.

Finally, it is known that silver sulfadiazine and other antimicrobial agents play a vital part in controlling bacterial infection in burn and other wound victims. Recently, finding alternative methods for fighting bacterial colonization are of rising concern, in coordination with the rise of antibiotic resistance. Delivery of silver in a controlled and sustained fashion is critical to its efficacy; studies have shown that the optimal wound concentration of silver lies between 20 μg/ml and 60 μg/ml. Exposure to silver too rapidly, however, can lead to the metabolism of silver compounds and ultimately cell death. Synthesized alginate microparticles combine the anti-bacterial effects of silver sulfadiazine with the absorption and slow-release capabilities of alginate.

Calcium alginate microparticles were successfully synthesized using a method involving emulsification and internal gelation. Alginate microparticles were created with and without the encapsulation of silver nanoparticles (SNP) and silver sulfadiazine (SSD). Concordant with previously mentioned research, the mean diameter of alginate microparticles alone and alginate microparticles containing SSD was 3.3±3.2 μm (standard deviation SD) and 6.9±6.5 μm (SD), respectively. Engineered microparticles were shown to be stable with regard to density, size and appearance over time. Additionally, alginate microparticles containing SSD were shown to have significant antimicrobial activity when tested against Staphylococcus epidermidis, a substitute for the pathogenic Staphylococcus aureus, at both 24 and 48 hours. The results demonstrated that the alginate microparticles have the ability to deliver and release the encapsulated SSD. Further testing utilizing an enzyme that breaks down alginates revealed that the release of SSD is sustained over a 48 hour period.

### EXAMPLES

The present embodiments will be described in greater detail by way of the following examples. The following examples are offered for illustrative purposes, and are not intended to limit the present embodiments in any manner. Those of skill in the art will readily recognize a variety of noncritical parameters which can be changed or modified to yield essentially the same results.

Alginate microparticles were created using an emulsification and internal gelation technique. The above technique was modified to encapsulate silver sulfadiazine (SSD) and silver nanoparticles (SNP).

Equipment and reagents used included sealant tape, incubator set at 37° C., lab egg mixer, Osterizer blender, photo-microscope, microscope slides and cover-slips, pH meter, fume hood, hemocytometer, a microwave, Petri dishes, q-tipped applicators, blank discs, streptomycin control disc (10 μg), laboratory glassware, pipettes, droppers, Tryptic Soy broth (dehydrated), nutrient rich agar, 70% ethyl alcohol, alginate lyase, hypochlorite solution, alginic acid sodium salt, 1M HCl, 1M NaOH, certified organic canola oil, glacial acetic acid, calcium chloride, calcium carbonate powder, 30 μm sieve filter, tween, Safranin O, silver sulfadiazine, silver nanoparticles in sodium citrate buffer (10 nm), and a Staphylococcus epidermidis (obtained from Carolina Biological).

The synthesized microparticles were analyzed for size using a photo-microscope and density using a hemocytometer. They were also analyzed for stability (taking into account size, density and appearance overtime). The engineered microparticles were tested for antimicrobial activity against Staphylococcus epidermidis cultured in Tryptic Soy broth and plated on nutrient rich agar plates Antimicrobial analysis of created particles involved the comparison of alginate microparticles alone, alginate microparticles containing SSD, alginate microparticles containing SNP, SSD alone, silver nanoparticles alone, blank discs, and an antibiotic control (Streptomycin) at 24 and 48 hours. Additionally, antimicrobial activity of alginate microparticles containing SSD was tested in conjunction with alginate lyase.

### Example 1

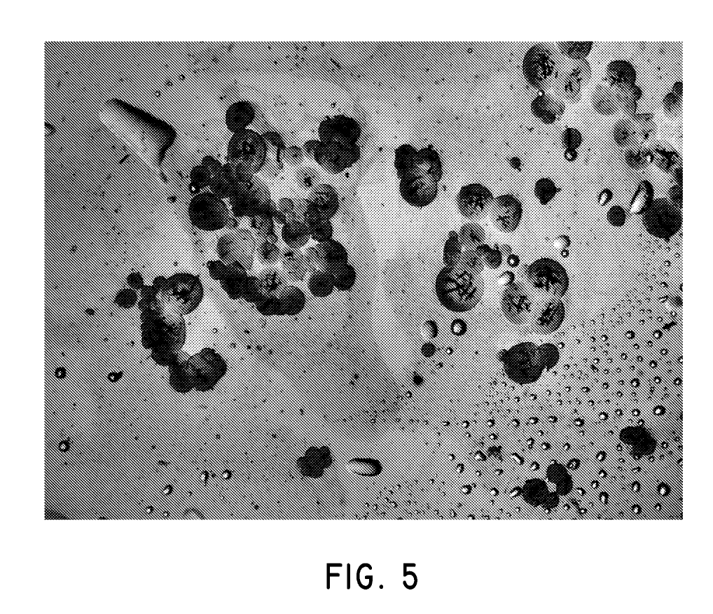

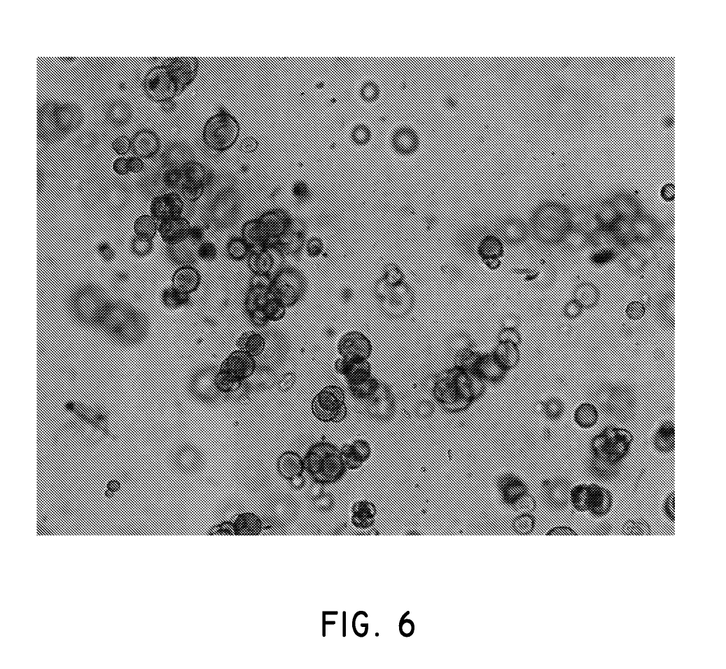

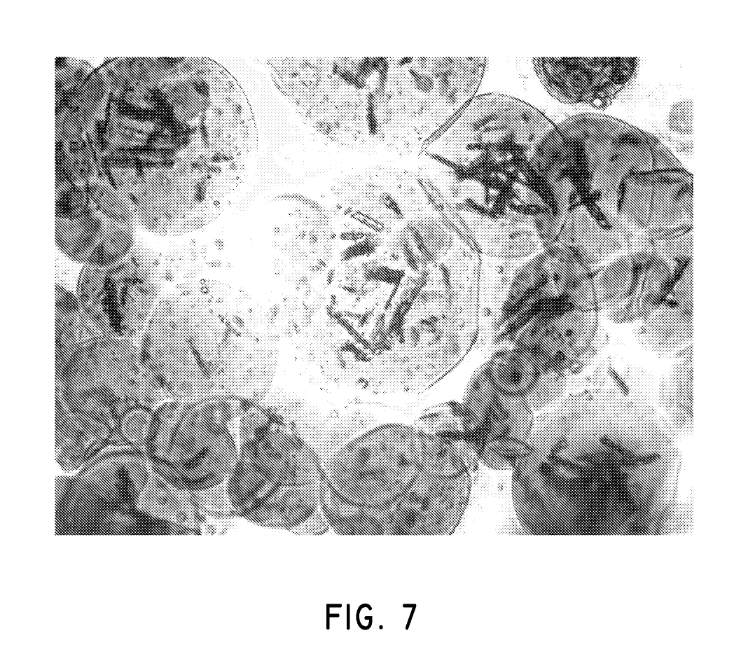

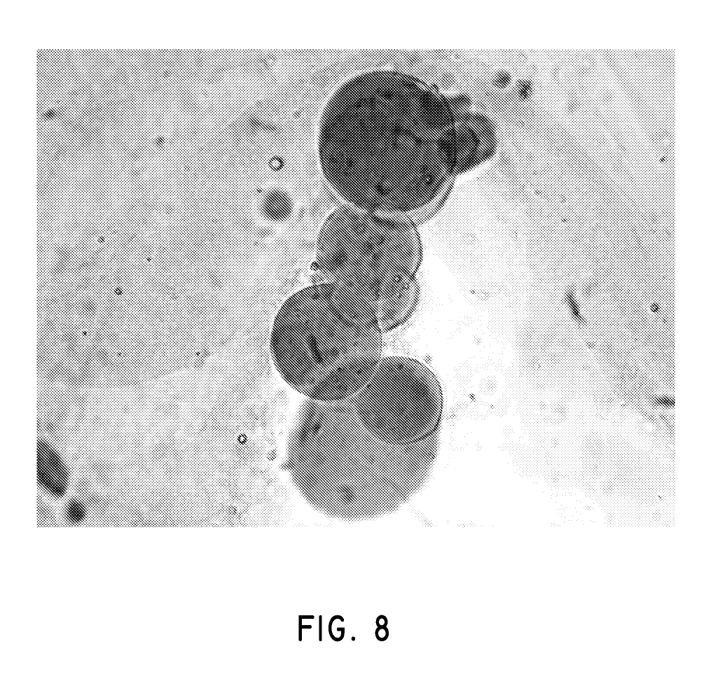

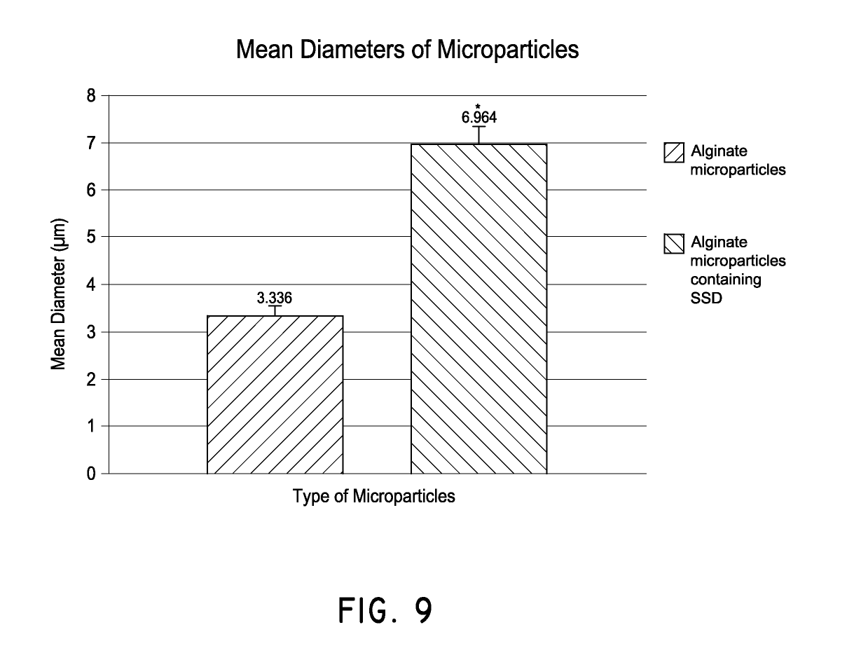

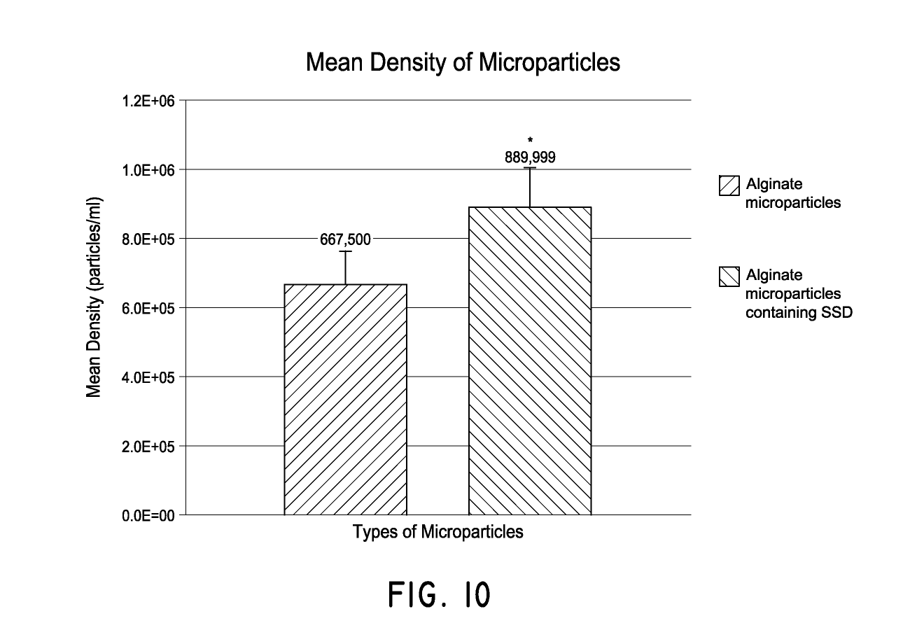

Alginate microparticles were successfully synthesized through a method involving emulsification and internal gelation. The created particles were shown, via photographic analysis and antimicrobial activity, to successfully encapsulate silver sulfadiazine (SSD). Alginate microparticles alone were shown to have a mean diameter of 3.3±3.2 μm (standard deviation SD). Additionally, the diameters of synthesized alginate microparticles were not significantly different between batches (p<0.05). Alginate microparticles containing SSD were shown to have a mean diameter of 6.9±6.5 μm (SD). The mean diameters of alginate microparticles containing SSD appeared to be significantly larger when compared to alginate microspheres alone (p<0.005) (FIG. 9). With regard to density, alginate microparticles alone were shown to have a mean density (in number of particles/ml) of 6.7×10⁵±3.3×10⁵ (SD). There was no observed statistical difference between batches (p<0.05). Alginate microparticles containing SSD had a mean density of 8.9×10⁵±3.9×10⁵ particles/ml (SD). Once again, alginate microparticles containing SSD appeared to be significantly more dense when compared to alginate microparticles alone (p<0.1) (FIG. 10). Photographic analysis of all the particles revealed that synthesized particles were spherical in shape (FIGS. 5-8). The engineered particles were shown to be stable with regard to mean density (p<0.05), size (p<0.05) and shape over the time period tested at 4° C. Furthermore, analysis of the alginate microparticles containing SSD showed visible amounts of SSD entrapped within the spheres (FIGS. 5 and 7).

### Example 2

Created microparticles were also tested over time to determine stability while in storage at 4° C. Results from density analysis showed that the mean density of alginate microparticles did not change significantly over a period of three weeks (p<0.05). Recorded outcomes also showed that the mean size of alginate microparticles did not significantly change over a three week period (p<0.005). Alginate microparticles containing SSD had similar characteristics; they did not change in regards to mean size or density over a period of two weeks (p<0.05). Photographic analysis of the alginate microparticles alone and alginate microparticles containing SSD at three and two weeks respectively showed that shape was consistent. Both synthesized particles appeared to be spherical and visible amounts of SSD could be seen encapsulated within the alginate microparticles.

### Example 3

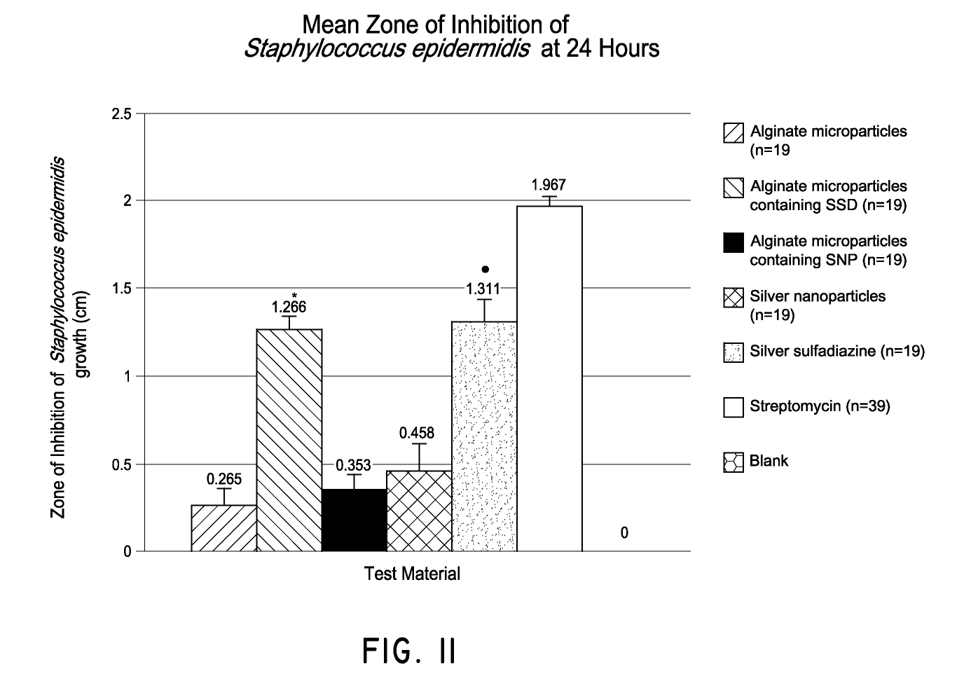

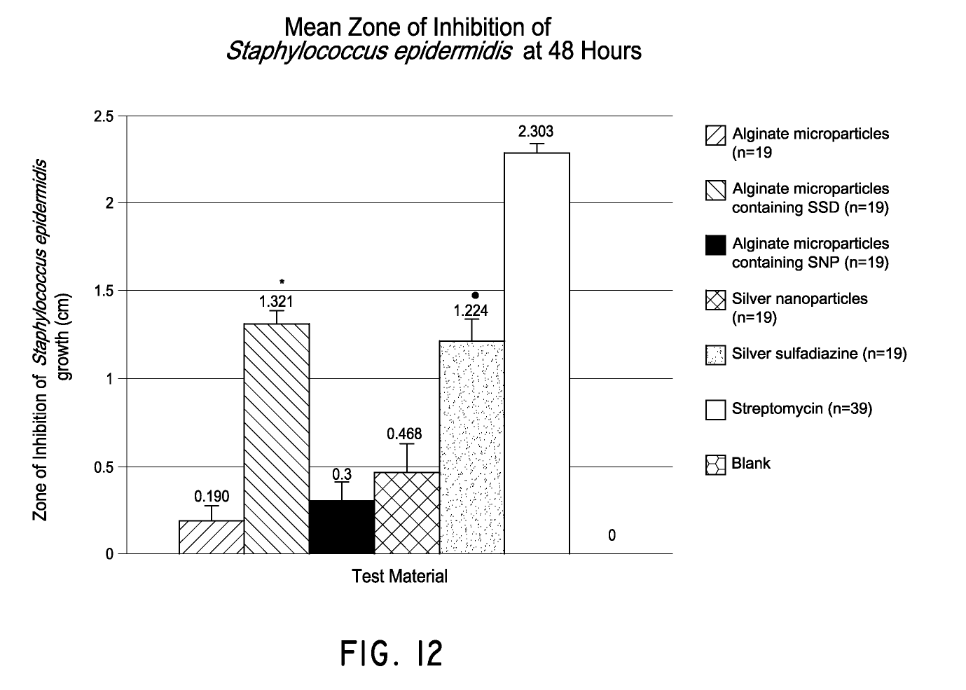

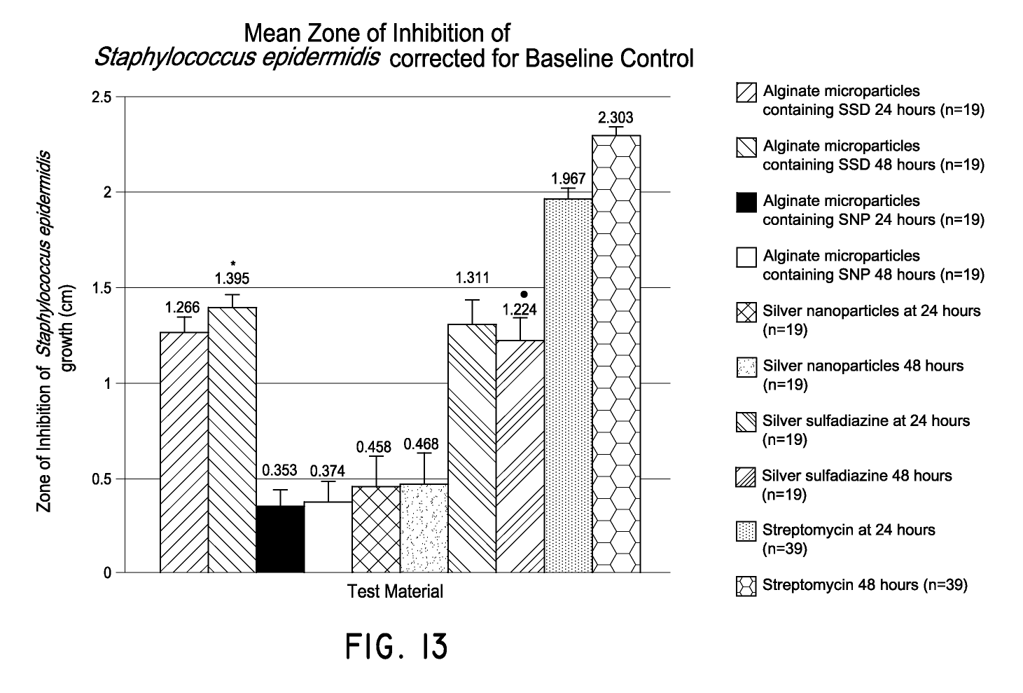

The synthesized microparticles were also tested for their antimicrobial activity against Staphylococcus epidermidis, a substitute for the pathogenic Staphylococcus aureus. In all test plates, the positive control was Streptomycin and this provided a large, consistent zone of inhibition whereas the blank disc showed no antimicrobial activity. Results showed that alginate microparticles containing SSD produced significantly increased antimicrobial activity when compared to alginate microparticles alone at both 24 and 48 hours (p<0.001) (FIGS. 11 and 12, respectively). Moreover, alginate microparticles containing SSD (undiluted) demonstrated statistically significant increased antimicrobial activity when compared to alginate microparticles containing SSD at concentrations 1:10 and 1:100 at 24 and 48 hours (p<0.001). Results demonstrated that there was a significant increase in the zone of inhibition of alginate microparticles containing SSD from 24 to 48 hours when the difference in the baseline control (alginate microparticles) was removed (p<0.05) (FIG. 12). Silver sulfadiazine alone, however, showed no significant increase in the zone of inhibition from 24 to 48 hours (FIG. 13). To add, at 24 hours there was no significant difference between SSD on its own and alginate microparticles containing SSD (p<0.05) (FIG. 12). At 48 hours, however, alginate microparticles containing SSD showed a larger zone of inhibition when compared to SSD alone (p<0.05) (FIG. 13).

### Example 4

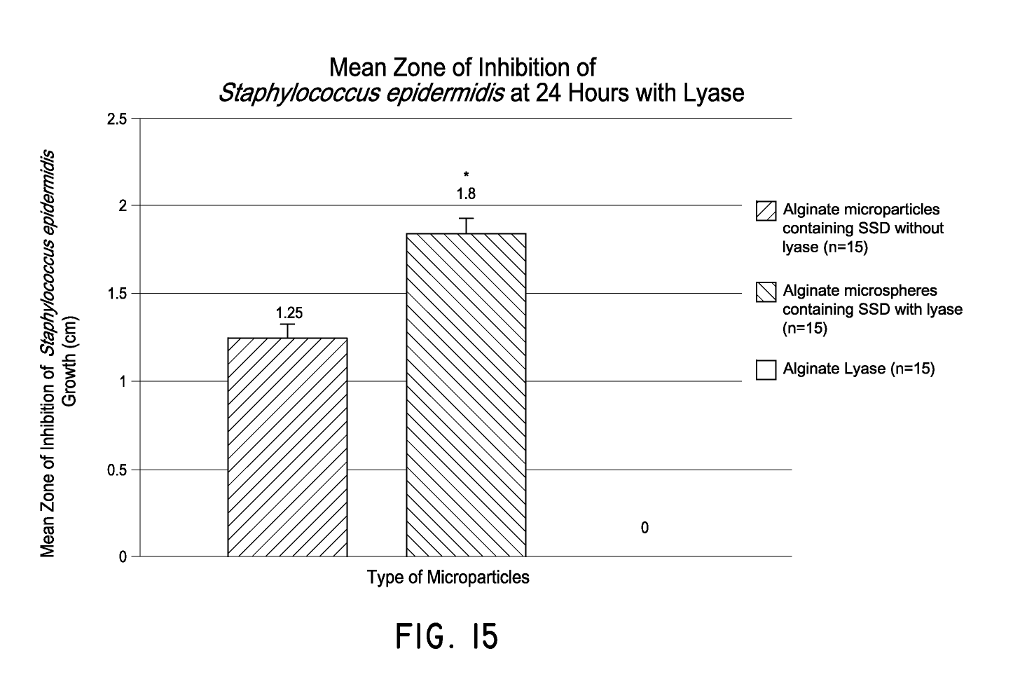

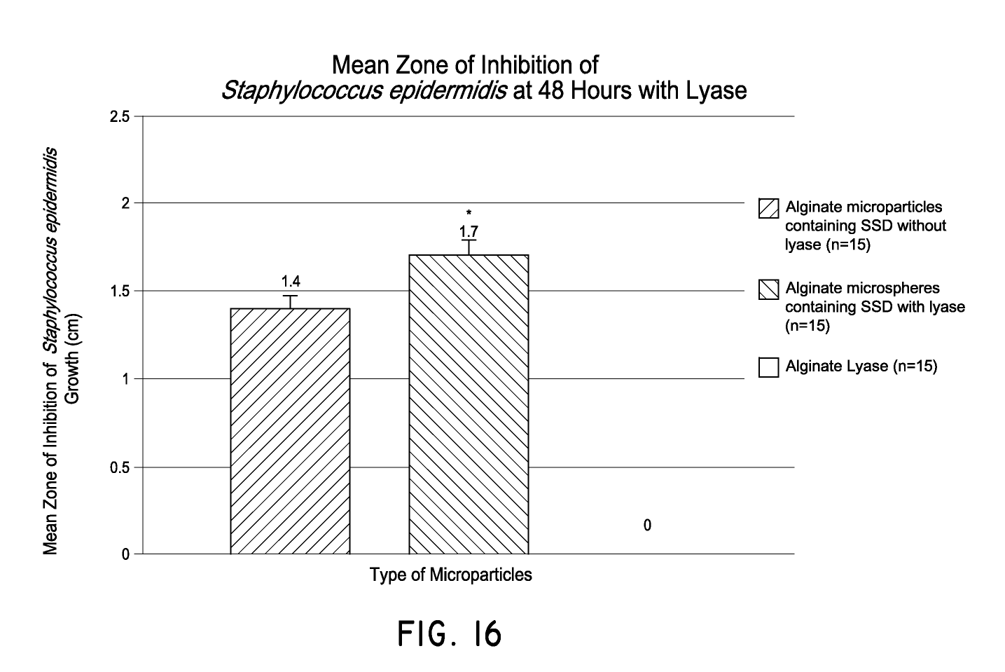

Experiments to determine antimicrobial activity were also conducted that incorporated the use of alginate lyase, an enzyme that breaks down alginate. Results showed that at both 24 and 48 hours there was a significantly larger zone of inhibition when comparing alginate microparticles containing SSD to alginate microparticles alone (p<0.001). Furthermore, alginate microparticles containing SSD and lyase were shown to have a significantly larger zone of inhibition when compared to alginate microparticles containing SSD without lyase (p<0.0001) at 24 and 48 hours (FIGS. 15 and 16). Alginate microspheres containing SSD and lyase were not shown to be significantly different between 24 and 48 hours (p<0.05).

It is to be understood that the above description is intended to be illustrative and not restrictive. Many embodiments will be apparent to those of skill in the art upon reading the above description. The scope of embodiments should, therefore, be determined not with reference to the above description, but should instead be determined with reference to the appended claims, along with the full scope of equivalents to which such claims are entitled. The disclosures of all articles and references, including patent applications, patents, PCT publications, and Genbank Accession Nos., are incorporated herein by reference for all purposes.

What is claimed is:

1. A composition comprising an alginate microparticle comprising silver, wherein said alginate microparticle has a mean diameter of less than 10 μm.

2. The composition of claim 1 wherein said alginate microparticle comprises an alginate lyase.

3. The microparticle population of claim 1 wherein said mean diameter is about 7 μm.

4. The microparticle population of claim 1 wherein said microparticle population is stable with regard to particle size and appearance after 2 weeks of storage at 4° C.

5. A method of delivery of an antiseptic comprising providing a composition comprising a microparticle population; said microparticles comprising an alginate and silver; wherein said microparticle population has a mean diameter of less than 10 μm.

6. The method of claim 5, wherein said microparticles further comprise an antibiotic or bacteriostatic.

7. The method of claim 5 wherein said silver comprises silver sulfadiazine, silver nano particles and/or colloidal silver.

8. The method of claim 5 wherein said composition provides sustained release of said silver from said alginate microparticle.

9. The method of claim 6, wherein said composition provides sustained release of said antibiotic.

10. The method of claim 8 wherein said sustained release continues for not less than 24 hours.

11. The method of claim 5, further comprising:
contacting a wound or infection site of a subject in need thereof with said composition comprising the microparticle population.

12. The method of claim 5, further comprising providing an alginate lyase.

13. The method of claim 5, wherein said composition comprises alginate lyase.

14. The method of claim 12 wherein said lyase releases substantially all of said antiseptic from said microparticle population.

15. A method of controlling a duration of exposure to an antiseptic, in a wound site of a subject, comprising:
providing a composition comprising a microparticle population, wherein said microparticle population comprises microparticles comprising an antiseptic, and wherein said microparticle population has a mean diameter of less than 10 μm;
measuring an amount of time during which a subject is to be exposed to said antiseptic, antibiotic or bacteriostatic; and
providing at the passage of said duration of time an alginate lyase to said wound site of said subject.

16. The method of claim 15 wherein said antiseptic, antibiotic or bacteriostatic comprises silver.

17. The method of claim 16 wherein said antiseptic, antibiotic or bacteriostatic comprises silver sulfadiazine, colloidal silver and/or silver nano particle.

18. The method of claim 15 wherein said concentration of said antiseptic provided is between 20 μg/ml and 60 μg/ml.
